(19) United States
(12) Patent Application Publication
(10) Pub. No.: US 2008/0041402 A1
(43) Pub. Date: Feb. 21, 2008

(54) Treatment of muscular imbalance in children by rerouting antagonist groups

(76) Inventor: Rahul K. Nath, Houston, TX (US)

Correspondence Address: THE MATTHEWS FIRM, 2000 BERING DRIVE, SUITE 700, HOUSTON, TX, 77057, US

(21) Appl. No.: 11/723,802

(22) Filed: Mar. 20, 2007

Related U.S. Application Data
(60) Provisional application No. 60/784,166, filed on Mar. 21, 2006.

(51) Int. Cl. A61B 19/00

(52) U.S. Cl. 128/898

(57) ABSTRACT

Following a diagnosis of muscle imbalance resulting from an injury to the Brachial Plexus, and a determination of which muscle groups have been weakened, tendons of some of the non-weakened muscle groups are transferred, for example, by suturing the tendons of one or more of the non-weakened muscle groups to one or more of the tendons of the weakened muscle groups, thereby tending to eliminate or decrease the muscle imbalance.

### RELATED APPLICATION

This application claims priority under U.S. Provisional Application No. U.S. 60/784,166, filed on Mar. 21, 2006.

### BACKGROUND OF THE INVENTION

All too often, children experiencing a brachial plexus injury, Erb's Palsy, experience a muscle imbalance, frequently associated with primary surgery undertaken to address the brachial plexus injury.

The brachial plexus is an arrangement of nerve fibers (a plexus) running from the spine (vertebrae C5-T1), through the neck, the axilla (armpit region), and into the arm. The brachial plexus is responsible for cutaneous and muscular innervation of the entire upper limb, with two exceptions: the trapezius muscle innervated by the spinal accessory nerve and an area of skin near the axilla innervated by the intercostobrachialis nerve.
The five roots are the five anterior rami of the spinal nerves, after they have given off their segmental supply to the muscles of the neck.
These roots merge to form three trunks:
   4“superior” or “upper” (C5-C6)
   “middle” (C7)
   “inferior” or “lower” (C8-T1)
Each trunk then splits in two, to form six divisions:
   anterior division of the superior, middle, and inferior trunks
   posterior division of the superior, middle, and inferior trunks
These six divisions will regroup to become the three cords. The cords are named by their position in respect to the axillary artery.
   The posterior cord is formed from the three posterior divisions of the trunks (C5-T1)
   The lateral cord is the anterior divisions from the upper and middle trunks (C5-C7)
   The medial cord is simply a continuation of the lower trunk (C8-T1)

Brachial Plexus injury, or Erb's Palsy, can be severe and permanent. This has been generally handled through management protocols, such as physical therapy and electrical stimulation.

Generally, if there is continued significant shoulder, elbow or hand weakness at 4 to 6 months after injury, brachial plexus surgical exploration is sometimes indicated.

The most commonly injured elements of the brachial plexus in children are the upper roots. These injuries often lead to loss of abduction and external rotation of the shoulder due to deltoid, supraspinatus, infraspinatus and teres minor denervation. This results in a muscle imbalance that eventually causes contractures to develop about the shoulder joint. Surgery has been used in the treatment of these contractures with great success in the restoration of shoulder abduction. However, the internal rotation posture in certain cases may have an additional component of bony rotation. This has significant functional consequences including limb shortening, loss of supination, winging of the scapula and lateral angulation of the elbow.

Traditionally, the approach to this problem has been the use of derotational humeral osteotomy, i.e., the treatment involving merely the manipulation of the involved bones and the spine to neutralize the hand and forearm. This approach attempts to restore the arm to a neutral position and often does do so. It does not address the primary cause of internal rotation, so further management may be necessary.

The need for secondary surgery does not mean that the primary surgery failed. It describes another type of surgery done later in time used to correct muscle imbalances that occur as a normal result of having a brachial plexus injury.

The situation of muscle imbalance is very common especially among those who have upper trunk (Erb's palsy) injuries. The majority of children whose injury does not completely resolve by 3 to 4 months of age will end up with a specific series of arm restrictions caused by a muscle imbalance between injured and uninjured muscles.

At the same time, the internal rotators (muscles that turn the arm and palm inward) and adductors (muscles that pull the arm to the side) of the arm are not involved in the injury because they are supplied by the lower roots of the plexus. Therefore, these strong muscles overpower the weak muscles and over time the child cannot lift the arm over the head or turn the palm out, because of the muscle imbalance.

### BRIEF DESCRIPTION OF DRAWINGS

FIG. 1 is an elevated, pictorial front view of a human, illustrating the muscles typically present in such humans.

FIG. 2 is an elevated, pictorial, back view of the human illustrated in FIG. 1; and

FIG. 3 is an elevated, pictorial view of a human hand, illustrating some of the muscles-tendon links found in such hands.

As an integral part of this secondary surgery in this present invention, designed to address contractures (tightness) in the axilla and in the chest, the antagonist groups are rerouted to improve function and global abduction.

### DETAILED DESCRIPTION OF THE PREFERRED EMBODIMENTS OF THE INVENTION

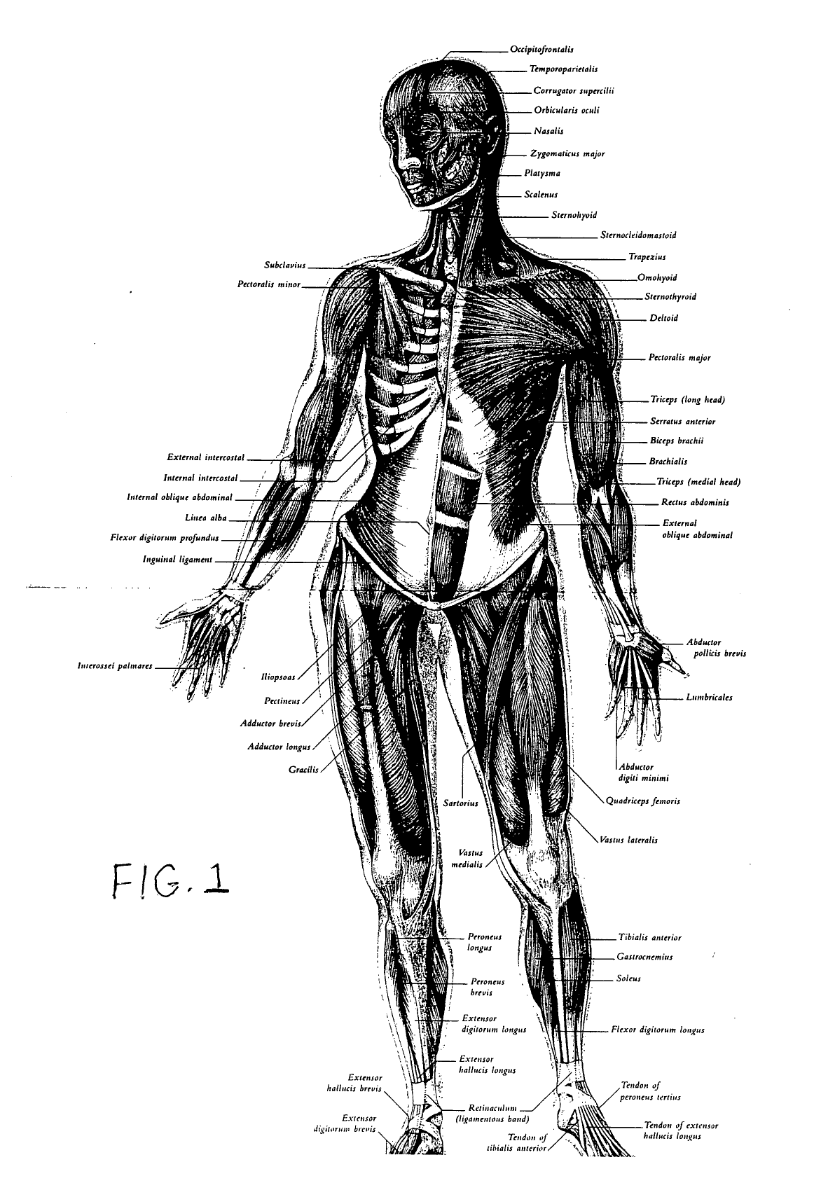

Referring now to FIGS. 1, 2 and 3 of the drawing, they are specific illustrations of the major muscles found in the human body, many of which can have their tendons transferred to the tendons of weakened muscles in accordance with the present invention.

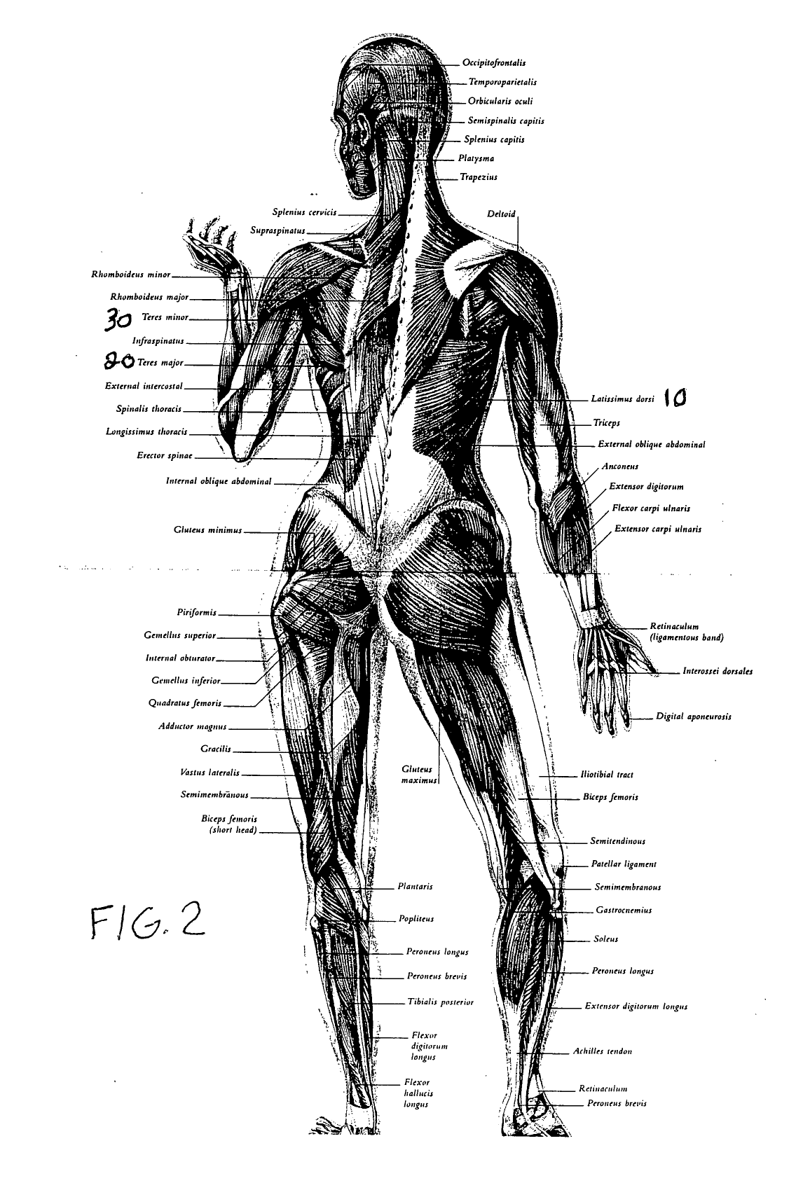

As but one example of the rerouting of the antagonist groups according to the invention, the Latissimus Dorsi 10 and the Teres Major 20 illustrated in FIG. 2 are each rerouted, i.e., transferred to the Teres Minor 30 illustrated in FIG. 2.

As used herein, the “antagonist” groups are the strong muscles and tendons, not involved in the original injury, which overpower the weak muscles and tendons and create the muscle and tendons imbalance.

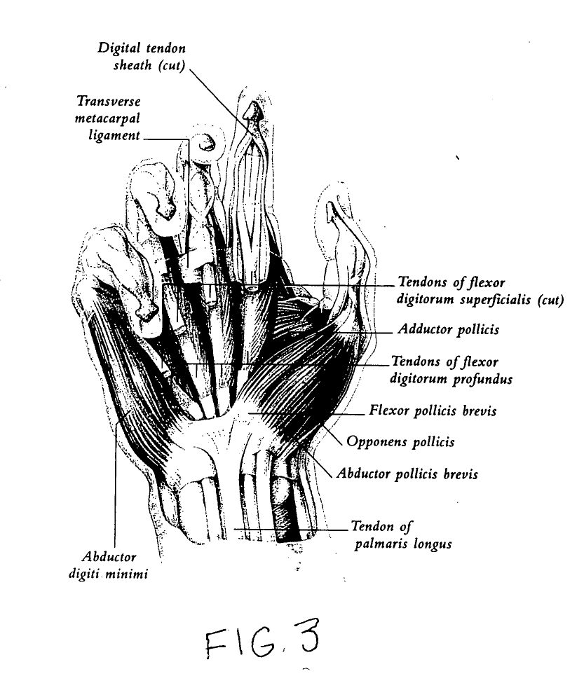

As yet another example of the rerouting of the antagonist groups, it should be appreciated that wrist, finger and thumb movements are often affected, leading to problems with hand grasp and finger pinch. A series of tendon/muscles transfers identified in FIG. 3 can help to achieve excellent function with these injuries as well. The basic principle is that some strong muscles and tendons can be rerouted to strengthen weaker functions by sewing the transferred tendons into the paralyzed ones.

### Example of Surgery According to the Invention

PREOPERATIVE DIAGNOSIS: Severe left brachial plexus injury with secondary and tertiary complications.

Postoperative Diagnosis:

1. Tight contractures of latissimus dorsi muscle.

2. Tight contractures of left teres major muscles.

3. Contracture of the left subscapularis muscle.

4. Contracture of the left triceps.

5. Compression of left axillary nerve.

6. Adduction deformity.

7. Weakness of left triceps with elbow flexion contracture.

Titles of Procedures:

1. Release of left latissumus dorsi contracture.

2. Release of teres major contraction.

3. Partial release of subscapularis contracture with lengthening.

4. Partial release of triceps fascia.

5. Decompression of axillary nerve.

6. External neuroplasty of axillary nerve.

7. Tendon transfer, latussimus dorsi tendon to teres minor.

8. Tendon transfer, teres major to teres minor.

ANESTHESIA: General endrotracheal

INDICATIONS FOR OPERATION: This child had suffered a severe left brachial plexus injury at the time of birth. The child was left with secondary and tertiary contracture of the axilla and chest. This had inhibited abduction to 80 degrees. Also present was a flexion contracture of the elbow with generalized weakness of the left upper extremity. These problems had not responded to conservative management and the child was therefore scheduled for surgery. The parent were counseled preoperatively about the risks and benefits and agreed to proceed with the surgery.

OPERATIVE NOTE: The child was brought to the operating room for general anesthesia. The left arm and chest were prepped and draped in the usual sterile fashion. An incision was created in the axilla and the latissimus dorsi muscle identified. Dissection now proceeded medially to the latissimus dorsi into the subscapularis fossa. The subscapularisis muscle was lengthened in a medial to lateral direction using electrocautery. Meticulous hemostasis was observed throughout. Dissection now proceeded along the latissimus dorsi tendon and the tendon was detached sharply from its insertion into the humerus. Similarly, the teres major tendon was separately released. Both tendons were now dissected inferiorly towards the inferior part of the scapula.

The axillary nerve was noted to be pinched and compressed beneath the triceps fascia. The triceps fascia was released and using meticulous microsurgical technique, the axillary nerve was decompressed. This major nerve decompression should allow improved function in the supplied muscle, the deltoid, and take off pressure on the axillary nerve. The axillary nerve was further externally neurolysed to remove external scar tissue. This major nerve neuroplasty should improve conduction in the axillary nerve and increase shoulder movement and stability. Stimulation of the nerve showed active movement of the deltoid muscle.

Attention was now turned to the teres minor tendon at the rotator cuff. An incision was created here. The tendons of the latissimus dorsi and teres major were separately sutured into that incision into that teres minor tendon. This was to improve external rotation and abduction of the arm.

The wound was thoroughly irrigated with antibiotic saline and meticulous hemostasis was confirmed. The wound was closed in two layers in absorbable suture over drain. Dry dressings were applied. The patient was awake and alert and extubated without complications following surgery. There were no complications. Blood loss was minimal.

1. In a process for treating muscular imbalance resulting from injury to the brachial plexus, the method steps comprising:
diagnosing which muscle groups have been weakened by the injury to the brachial plexus, thereby differentiating the weakened muscle groups from the non-weakened muscle groups; and
transferring one or more of the tendons from the non-weakened muscle groups into the tendons of the weakened muscle groups, thereby at least partially restoring muscle balance between the weakened and non-weakened muscle groups.

2. The process according to claim 1, wherein the tendons of the Latissimus Dorsi are transferred to the Teres Minor.

3. The process according to claim 1, wherein the tendons of the Teres Major are transferred to the Teres Minor.

4. The process according to claim 1, wherein the tendons of each of the Latissimus Dorsi and Teres Major are both transferred to the Teres Minor.
